(19) United States
(12) Patent Application Publication
(10) Pub. No.: US 2006/0293571 A1
(43) Pub. Date: Dec. 28, 2006

(54) Distributed architecture for remote patient monitoring and caring

(75) Inventors: Wanqun Bao, San Jose, CA (US); Wen Huang, Cupertino, CA (US)

Correspondence Address: STAINBROOK & STAINBROOK, LLP, 3558 ROUND BARN BLVD., SUITE 203, SANTA ROSA, CA, 95403, US

(73) Assignee: SKANDA SYSTEMS, SAN JOSE, CA (US)

(21) Appl. No.: 11/166,813

(22) Filed: Jun. 23, 2005

(51) Int. Cl. A61B 5/00

(52) U.S. Cl. 600/300; 128/920

(57) ABSTRACT

The present invention provides a system and method for remote patient monitoring and caring, wherein the system has a distributed architecture. The system comprises sensors for measuring real-time physiological parameters, such as ECG and SpO₂, from patients. The system also comprises a monitoring terminal for monitoring these parameters. A local processing station analyzes the real-time physiological parameters. A central processing system processes the real-time physiological parameters and their analysis, in combination with the relevant historical medical data, for each patient. The medical staff uses this processed data to provide required medical care to the patients. Further, the system provides remote access of patients' data, via remote access stations

### BACKGROUND OF THE INVENTION

1. Field of the Invention

The present invention relates generally to a method and a system for patient monitoring and caring. The present invention specifically relates to a distributed architecture for remote patient monitoring and caring.

2. Description of the Related Art

Remote patient monitoring and caring provides a solution to the problem of shortage of trained medical professionals. It allows efficient usage of the time and resources of the trained medical professionals, thereby, enabling more patients to receive the required care and medical aid.

Seriously ill patients very often require constant monitoring and care. Dedicating a medical professional to each such patient requires significant resources, and is not always possible. Remote patient monitoring systems allow for continuous monitoring of such patients.

In a remote patient monitoring system, the real-time physiological data collected from a patient is monitored at a physically separate location. It allows a medical professional to provide care to patients, who are not located in his/her physical vicinity. Additionally, a single medical professional can provide care to more than one patient at a given time.

Various patient monitoring systems exist in the art. One such system is described in the US patent application number 20040068195 titled, ‘Method and Apparatus for Wearable Digital Wireless ECG Monitoring’, Louis Massicotte, et al. The patent application relates to a wearable digital wireless electrocardiogram (ECG) monitoring system. The patent publication mentions that the monitoring system comprises two parts; a wireless digital ECG and a wireless central device. The physiological data of the patient collected is in the form of a full ECG curve and is digitized. After being digitized, the signal is sent wirelessly to a central module that analyzes this data.

Another system is described in U.S. Pat. No. 6,544,173, titled ‘Patient Monitoring System’, Welch Allyn Protocol, Inc., Kenneth G. West, et al. This patent relates to a patient monitor, which uses a wired communication system to connect sensors for measuring the physiological parameters of a patient. The system further comprises a central station, adapted to establish communications with the patient monitor via a wireless transceiver, and to analyze the patient's physiological parameters from the patient monitor.

Yet another system is described in the US patent application number 20010023315, titled, ‘Cellular Architecture and Data Transfer Methods for Real-time Patient Monitoring within Medical Facilities’, Terry Flach, et al. The patent publication relates to a system for collecting the real-time physiological data of patients of a medical facility, and for transferring the data via Radio Frequency (RF) to a real-time data distribution network for monitoring and display. The system comprises wireless remote telemeters which attach to respective patients, and which collect and transmit the physiological data of the patients. Further, the remote telemeters also communicate bi-directionally with a number of ceiling-mounted RF transceivers, referred to as ‘VCELLs,’ using a wireless Time Division/Demand Multiple Access (TDMA) protocol.

Another system is described in the U.S. patent publication number 20040102683, titled, ‘Method and Apparatus for Remotely Monitoring the Condition of a Patient’, Sukhwant Singh Khanuja, et al. The patent publication describes a system that provides a wireless communication link between a patient monitoring device, worn by a patient, and a local hub. The hub is configured to automatically transfer the data to a remote server, which can be a public or private communications network. The patent publication mentions that the system provides for enhanced functionality and allows trends of the physiological data to be selectively generated. Further, the patent publication mentions that any other person apart from the patient or the medical staff can set thresholds to automatically notify the patient or other third parties, by various communication methods, such as e-mails and pagers, when any particular physiological data exceeds a predetermined threshold.

Although, the above mentioned systems provide remote patient monitoring, one or more of these systems use wired communication between two or more of their components, making the system difficult to manage. Further, one or more of these systems deploy bulky devices which are not easy to use in everyday life. The form of wireless communication used by some of the above systems is also not secure. There is risk of intermingling of the physiological data of two patients. Yet another disadvantage of one or more of the above systems is that they work only in specific geographical areas, such as a medical centre or a hospital, which limits the scope of their usage. In light of the above discussion, it is apparent that there is a need for a system with a distributed architecture that is scalable, secure, cost-effective and user-friendly, and which can be deployed in different environments, such as medical and non-medical facilities.

### SUMMARY OF THE INVENTION

An objective of the present invention is to provide a system for remote patient monitoring and caring, wherein the system has a distributed architecture.

An objective of the present invention is to provide a system that can distribute the functionalities of the remote patient monitoring and caring system among different but functionally related components.

Another objective of the invention is to provide a method for making the communication system employed in the remote monitoring and caring system more secure.

Yet another objective of the present invention is to provide a bi-directional communication system between the various components of the remote monitoring and caring system, wherein the means of communication can be wired or wireless, and can be different between different components.

To achieve the above-mentioned objectives, in accordance with the purpose of the present invention, the present invention provides a method and system for remote patient care and monitoring through the use of secure means of communication.

The invention provides remote care to patients through the use of a system that comprises a plurality of sensors, monitoring terminals, local processing stations, central processing systems and remote access stations.

Sensors measure real-time physiological parameters, such as ECG, glucose and SpO₂, of a patient. The monitoring terminal collects these parameters from all sensors attached to a particular patient. The local processing station analyzes these real-time physiological parameters collected by the monitoring terminal. Finally, a central processing system carries out a further analysis of these real-time physiological parameters of patients, in combination with their relevant historical medical data. Medical staff use this processed data to provide timely and urgent medical care to patients. This system also provides remote access of a patient's data, via remote access stations.

The above-mentioned system and method can be deployed in medical as well as non-medical facilities. Further, the system provides bi-directional and secure, wired or wireless communication between its various components. Finally, all the functionalities of the system are distributed among the various components of the system.

### BRIEF DESCRIPTION OF THE DRAWINGS

So that the manner in which the above recited features of the present invention can be understood in detail, a more particular description of the invention, briefly summarized above, may be had by reference to various embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates a remote patient monitoring and caring system, in accordance with an embodiment of the invention;

FIG. 2 represents a sensor, in accordance with an embodiment of the invention;

FIG. 3 is an illustration of a monitoring terminal, in accordance with an embodiment of the invention;

FIG. 4 is an illustration of a local processing station, in accordance with an embodiment of the invention;

FIG. 5 is an illustration of a central processing system, in accordance with an embodiment of the invention;

FIG. 6 depicts a flowchart that describes a method for remote patient monitoring and caring, in accordance with an embodiment of the invention;

FIG. 7 illustrates the use of the remote patient monitoring and caring system for monitoring multiple patients in a community, such as a nursing home, in accordance with an embodiment of the invention; and

FIG. 8 illustrates the use of the remote patient monitoring and caring system for the monitoring a patient who is carried on a patient trolley, in accordance with an embodiment of the invention.

### DETAILED DESCRIPTION

The invention relates to the domain of telemedicine, which is defined by the American Telemedicine Association (ATA) as, ‘the use of medical information exchanged from one site to another via electronic communications for the health and education of the patient or health care provider, and for the purpose of improving patient care’.

The invention provides a method and system for remote monitoring and caring, wherein the system has a distributed architecture.

The remote patient monitoring and caring system collects real-time physiological parameters from the patients and analyzes them. These parameters and their analysis are processed along with the relevant medical history of the patients. The results of this analysis are used to deliver the required medical attention and care to the patients.

The system allows the medical staff to compare real-time physiological parameters of the patients with their medical histories. This allows the medical staff to get a complete perspective of the patients' medical condition, thereby, enabling them to provide efficient medical care and assistance to their patients.

Further, the remote monitoring and caring system allows the patients to contact medical staff, in case of an emergency, by sending an alarm signal. This allows the medical staff to provide immediate medical assistance and care.

All the components of the remote patient monitoring and caring system are connected via a bi-directional communication system. The bi-directional communication system not only allows the medical staff to monitor a patient's medical condition, but also allows the medical staff to provide care to the patient. An efficient two-way communication system between the patient and the medical staff is essential for taking care of the patient in an efficient manner.

The communication system is secure and reduces the risk of any intentional or unintentional breach of security during the exchange of data and information between the various components.

The communication between various components of the remote patient monitoring and caring system can be wired or wireless. The means of communication used can be different between different components. The choice of the means of communication depends on factors, such as the distance between the components, power consumption, and cost.

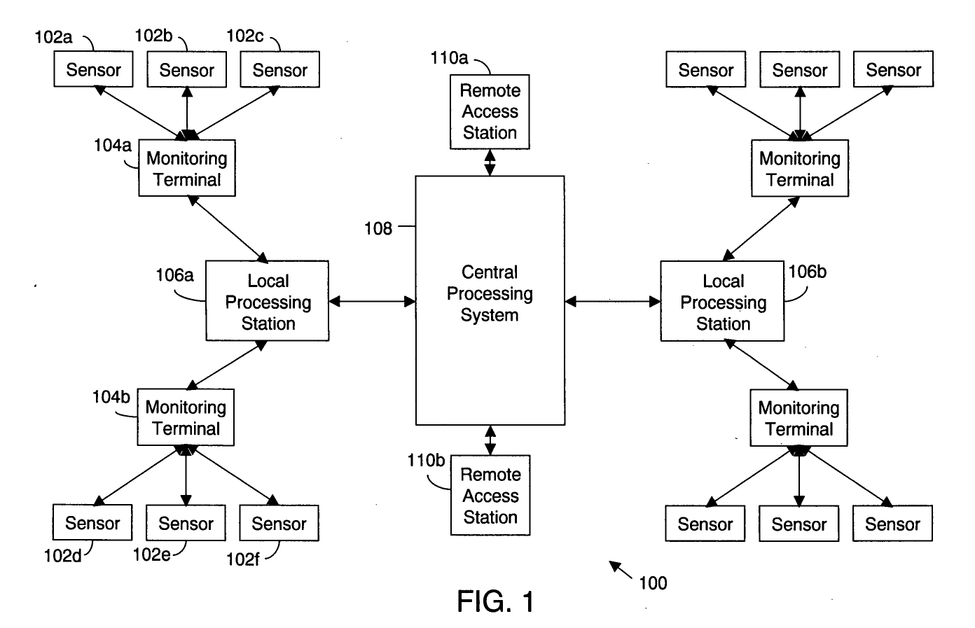

FIG. 1 illustrates a remote patient monitoring and caring system 100, in accordance with an embodiment of the invention. In an embodiment of the invention, remote patient monitoring and caring system 100 includes sensors 102a, 102b, 102c, 102d, 102e and 102f, monitoring terminals 104a and 104b, local processing stations 106a and 106b, a central processing system 108, and remote access stations 110a and

Remote patient monitoring and caring system 100 has a distributed architecture. The functionalities of remote patient monitoring and caring system 100 are distributed among its various components.

Sensors 102a-102f are medical domain-specific devices that measure the real-time physiological parameters of a patient. Some examples of the real-time physiological parameters that are measured by using sensors include, but are not limited to, ECG waveform, SpO₂, body temperature, and glucose level.

Monitoring terminals 104a and 104b collect real-time physiological parameters measured by all the sensors attached to a particular patient.

The monitoring terminals can be in the form of a device that can be worn by the patient. In an embodiment of the invention, the monitoring terminal is a wearable, watch-like, and lightweight device.

The real-time physiological parameters collected by monitoring terminals 104a and 104b are analyzed by local processing stations 106a and 106b connected to the patients.

Central processing system 108 further analyzes and processes the real-time physiological parameters analyzed by one or more local processing stations, connected to it. The central processing system combines the analysis of the real-time physiological parameters, along with the historical medical data of each patient, thus performing an analysis of the patients' medical condition. The central processing system also documents historical medical data of all patients, for future reference. Based on this analysis, remote patient monitoring and caring system 100 facilitates the medical staff to provide the required medical care and assistance to the patients.

The analysis performed at central processing system 108 may be automated, semi-automated or manual, depending upon the complexity and criticality of the analysis. Medical staff may be deployed in the vicinity of central processing system 108, that may manually view and analyze the data received from various components of the systems. Additionally, certain analyses may be automated according to certain rule-based systems.

Remote patient monitoring and caring system 100 continuously, periodically, or instantly monitors the conditions of the patients and provides regular feedbacks and updates to the patients and the medical staff. The patients can receive and view these feedbacks and updates via the monitoring terminal. The medical staff, which is responsible for providing the care and immediate aid to the patient, can receive and view these updates through specific communication devices, which may be mobile or static. For example, the medical staff can get regular updates on a specified computer terminal. Additionally, the updates can be provided in the form of text or voice messages over mobile phones or pagers.

Remote patient monitoring and caring system 100 is also capable of identifying and flagging a particular situation as emergency. This would be based on the analysis carried out by central processing system 108 or local processing station 106.

In case of an emergency or any discrepancy or irregularity in the measured physiological parameters, the system can send an alarm signal to the patient. Further, the system also sends an alarm signal to the medical staff taking care of the patient, who provides immediate attention and care to the patient.

Remote patient monitoring and caring system 100 can comprise more than one central processing system 108, depending on the number of patients that are being catered to.

It may not always be possible that the patient or the medical staff is in the vicinity of a device, such as a terminal, that is receiving regular updates from central processing system 108. To handle such situations, remote patient monitoring and caring system 100 allows remote access to the medical staff, via remote access stations 110a and 110b. The remote access stations allow the medical staff to view and analyze the real-time physiological parameters and the historical medical data of patients.

Remote access stations 110a and 110b provide remote access of the real-time physiological parameters and the historical medical data of a patient stored in central processing system 108. Remote access stations 110a and 110b may be present inside or outside the medical facility, in which remote patient monitoring and caring system 100 is deployed. The remote access stations allow access to the real-time physiological parameters and the historical medical data present in the central processing systems, to medical staff and other authorized persons, such as relatives and caretakers of the patient, present within or outside the medical facility. In an embodiment of the invention, the remote access stations are present within the medical facility in which the remote patient monitoring and caring system has been deployed. The local positioning of the remote access stations provides fast access to medical staff and other authorized persons. Thus, the medical staff can monitor multiple patients in real-time. Generally, only a web browser is required for the remote access station to function.

In an embodiment of the invention, remote patient monitoring and caring system 100 can be deployed in a hospital or a clinic. In another embodiment of the invention, remote patient monitoring and caring system 100 can be located in a non-hospital environment. In yet another embodiment of the invention, the patients connected to remote patient monitoring and caring system 100 can be located at home, while they are being monitored and cared for, from a remote medical facility. In this embodiment, various components of remote patient monitoring and caring system 100 are dispersed across various locations, such as the home of the patient/patients, homes for the elderly and hospitals.

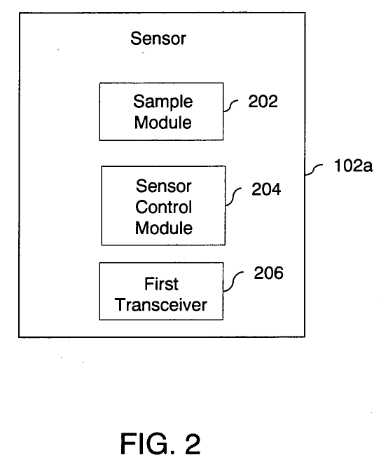

FIG. 2 represents sensor 102a, in accordance with an embodiment of the invention. In an embodiment of the invention, sensor 102a comprises a sample module 202, a sensor control module 204, and a first transceiver 206.

As mentioned earlier, sensor 102a measures the real-time physiological parameters of a patient. Sample module 202 is a domain-specific circuitry that measures the physiological parameter it is designed for. Further, sample module 202 filters any noise present in the real-time physiological parameters measured, and digitizes them. For example, if sensor 102a measures the ECG signals of a particular patient, it removes any noise occurring in the data measured and digitizes the data obtained.

Sample module 202 sends the digitized real-time physiological parameters to sensor control module 204. Sensor control module 204 formats the digitized real-time physiological parameters and transmits this formatted data to monitoring terminal 104 via first transceiver 206.

Sensor control module 204 is capable of changing the parameters that regulate sensor 102a in order to manage its efficiency and performance. Some examples of these parameters are sample rate, sampling time interval, and parameters for changing the configuration of sensor 102a.

In an embodiment of the invention, first transceiver 206 receives commands for changing the parameters that control sensor 102a, from the local processing station or the central processing system, via the monitoring terminal it is connected to. First transceiver 206 sends these commands to sensor control module 204. Based on these commands, sensor control module 204 can change the parameters that regulate sensor 102a.

In another embodiment of the invention, sensor 102a can be temporarily de-activated, when not in use.

Sensor 102a also includes a power source. The power source can be a low power consumption battery.

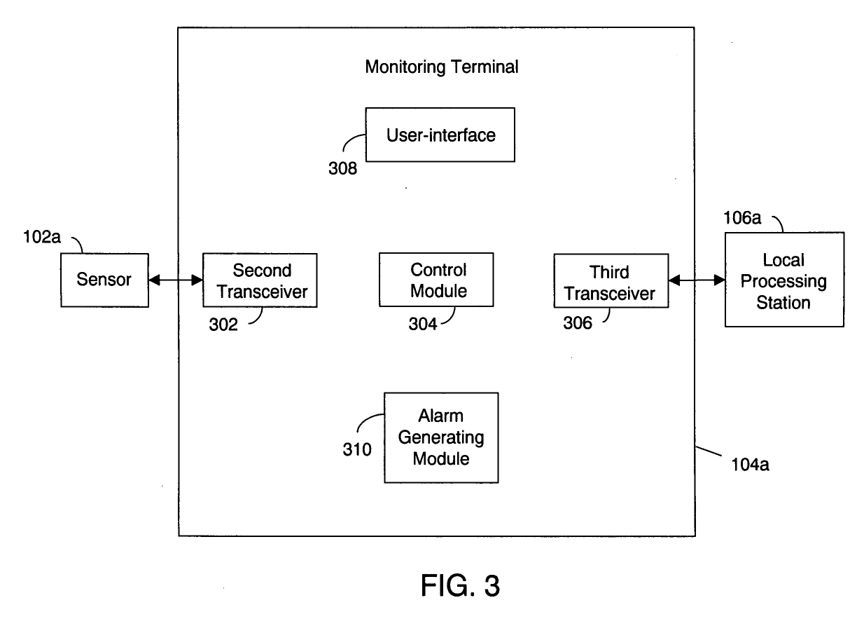

FIG. 3 represents monitoring terminal 104a, in accordance with an embodiment of the invention.

In an embodiment of the invention, monitoring terminal 104a comprises a second transceiver 302, a control module 304, a third transceiver 306, a user-interface 308, and an alarm-generating module 310.

Second transceiver 302 enables communication between monitoring terminal 104a and the sensors it is connected to. Second transceiver 302 receives digitized and formatted real time physiological parameters from the sensors monitoring terminal 104a is connected to.

Second transceiver 302 is capable of sending real-time physiological parameters, collected from the sensors, to control module 304 that formats these real-time physiological parameters. Formatting enables the arrangement of the real-time physiological parameters according to a pre-defined format. This format enables the local processing station and the central processing system to understand, analyze, and use the physiological parameters effectively. Second transceiver 302 is also capable of sending commands to the sensors, wherein these commands are related to the controlling and re-configuring the sensors.

Third transceiver 306 enables communication between monitoring terminal 104a and the local processing station it is connected to. Third transceiver 306 is capable of receiving messages sent by the central processing station to the patient, via the local processing station. These messages are sent to user-interface 308 that communicates these messages to the patient.

User-interface 308 includes input and output devices that enable patients and medical staff to communicate with each other. User-interface 308 enables a patient to view his/her real-time physiological parameters as well as feedbacks and updates sent by the central processing system. Further, user-interface 308 is capable of transmitting messages generated by the patient, via third transceiver 306, to the medical staff working at the central processing system. These messages can be in the form of, but not limited to, text, audio or video, and so forth.

User-interface 308 includes, but is not limited to, audio devices (e.g., speaker, microphone and alarm), video devices (e.g., LCD and camera) and data interpretation devices (e.g., LCD, keypad and push-button switches). In an embodiment of the invention, user-interface 308 includes buttons/keys for other functionalities, such as the monitoring terminals being manually switched off.

Alarm-generating module 310, present in monitoring terminal 104a, enables a patient to generate alarm signals to the medical staff, for immediate help.

The connection between the sensors and the monitoring terminals can be wired or wireless. Some examples of wireless communication technologies that can be used are short range, low data rate, wireless communication technologies, such as ZigBee and Bluetooth.

The connection between the monitoring terminals and the local processing stations can be wired or wireless. Some examples of wireless communication used between the monitoring terminal and the local processing station include, but are not limited to, Wireless Fidelity (WiFi), Global System for Mobile Communications (GSM), Bluetooth and ZigBee.

The monitoring terminal has two communication interfaces, one to the sensors and the other to the local processing station. Both these interfaces are bi-directional. The monitoring terminal sends the real-time physiological parameters of a particular patient collected from all sensors, to the local processing station. Further, the monitoring terminal also sends messages from the patient to the central processing system, via the local processing station it is connected to. These messages can be in text, audio or video form, and the like. These messages are used by patients to contact medical staff present at the central processing system. Further, the patient can also send alarm signals by using the monitoring terminal, to the central processing system, via the local processing station, whenever there is a need for immediate attention.

In an embodiment of the invention, a patient may be monitored via one or more monitoring terminals. Additional monitoring terminals are worn by caretakers or the medical staff responsible for the patient. For example, a doctor in charge of a patient may possess a monitoring terminal that monitors that patient. The monitoring terminal is regularly updated with the patient's physiological parameters and historical medical data, by the central processing system. Further, the doctor can send an alarm signal to the local processing station or the central processing station, via this monitoring terminal, in case any unusual symptoms are observed in the patient. Finally, the doctor can also obtain any required data related to the patient from the central processing system using his/her own monitoring terminal.

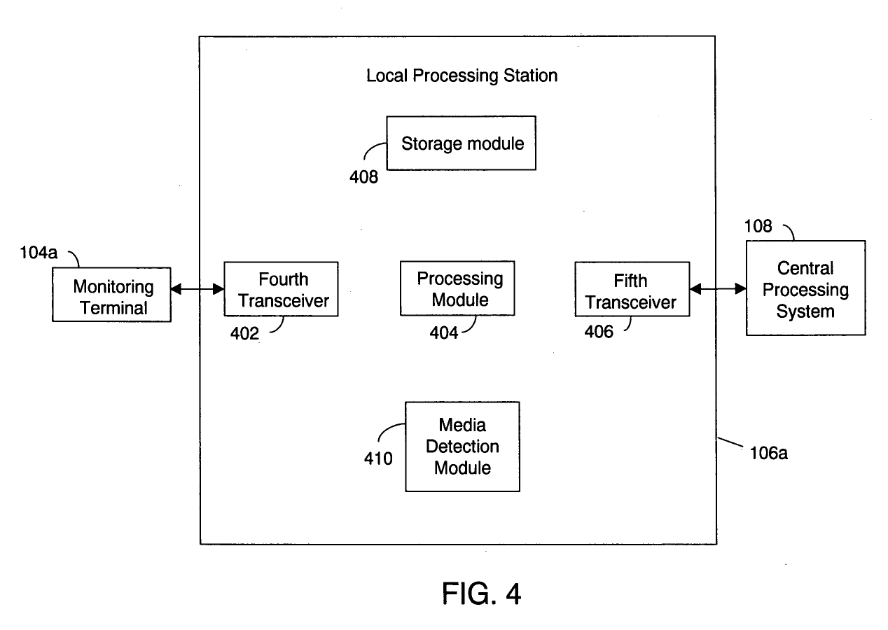

FIG. 4 represents local processing station 106a, in accordance with an embodiment of the invention. In an embodiment of the invention, local processing station 106a comprises a fourth transceiver 402, a processing module 404, a fifth transceiver 406, a storage module 408, and a media detection module 410.

Local processing station 106a can be connected to one or more monitoring terminals. For example, in a household, each member can have his/her own monitoring terminal that collects real-time physiological parameters from the sensors attached to his/her body. All the members of the household can share a common local processing station located in the same house. A similar deployment can be adopted in an environment, where people live in a community, such as a hospital, office, old-age home, hostel, hotel, or a clinic.

Fourth transceiver 402 enables communication between local processing station 106a and the monitoring terminals. Fourth transceiver 402 is capable of receiving real-time physiological parameters and messages from the monitoring terminals to which local processing station 106a is connected. Fourth transceiver 402 transmits the messages obtained from monitoring terminals to processing module 404. Further, fourth transceiver 402 also sends messages to the monitoring terminals of patients, wherein the messages are originally sent by the medical staff, via the central processing system it is connected to.

Fourth transceiver 402 also directs commands and instructions to the monitoring terminal, wherein these commands and instructions are either sent by the central processing system or generated by the monitoring terminal itself.

Processing module 404 performs a domain-specific analysis of the real-time physiological parameters received from each patient. This analysis includes basic trends that are generated from the real-time physiological parameters collected from each patient. For example, if processing module 404 receives data related to Electrocardiogram (ECG) waveforms, processing module 404 analyses this data and notes the abnormalities in the waveform. Processing module 404 transmits the analysis of all real-time physiological parameters it receives, to fifth transceiver 406.

In an embodiment of the invention, processing module 404 is capable of triggering an alarm signal based on a pre-defined logic. Examples of the pre-defined logic include abnormal fluctuations in the physiological parameters, and the physiological parameters having a value above or below an acceptable pre-defined range. The acceptable ranges for all physiological parameters and acceptable frequencies of fluctuation for all physiological parameters are stored in processing module 404. In an embodiment of the invention, these ranges and frequencies may be stored in a memory present in processing module 404. Further, these ranges and frequencies are either specific to a particular patient, based on their medical histories, or are pre-defined ranges and frequencies considered common for all patients.

Therefore, if processing module 404 finds any irregularity in the physiological parameters of a patient, during their analysis, processing module 404 can generate an alarm signal based on the stored ranges and frequencies. This alarm signal is sent to the patient and the medical staff stationed at the central processing system, via fourth transceiver 402 and fifth transceiver 406, respectively.

Fifth transceiver 406 enables communication between local processing station 106a and the central processing system it is connected to. Fifth transceiver 406 is capable of sending real-time physiological parameters and their analysis to the central processing system for further analysis. Further, fifth transceiver 406 receives messages for the patients from medical staff stationed at the central processing system.

Storage module 408 temporarily stores any data that is not being transmitted or analyzed, at a given time. Examples of stored data are real-time physiological parameters, text, audio and video messages, and so forth. In an embodiment of the invention, the storage module uses the Random Access Memory (RAM) and other storage devices, such as flash memory.

Media detection module 410 detects the means for transmission available for the transmission of any kind of data. In addition, media detection module 410 is responsible for determining the most suitable transmission means that should be used, in cases where more than one such means is available. This can be done by evaluating the quality of different wireless transmission means. Further, media detection module 410 connects to the determined means for transmission. The determination of the suitable transmission means may be based on a simple logic, wherein each transmission means is allocated a different priority, based upon the type of message. In an embodiment of the invention, media detection module 410 detects two different means for transmission available between local processing station 106a and the monitoring terminal, and local processing station 106a and the central processing system.

The connection for communication between the monitoring terminals and the local processing stations can be wired or wireless. Some examples of wireless communication technologies used between the monitoring terminal and the local processing station include, but not limited to, WiFi, GSM, Bluetooth or ZigBee.

The connection for communication between the local processing stations and the central processing systems can be wired or wireless. Some examples of wireless communication used between the local processing station and the central processing system include long range wireless communication technologies, such as GSM and WiFi.

The local processing station has two communication interfaces, one to the monitoring terminal and the other to the central processing system. Both these interfaces are bi-directional. These two-way communication interfaces may or may not use the same means for communication. The communication between the local processing station and the central processing station is enabled with a long-range technology, such as, but not limited to, WiFi and GSM. The communication between the local processing station and the monitoring terminal may or may not be enabled by using the same long-range technology, as being used between the local processing station and the central processing station. Short-range wireless technologies, such as, but not limited to, ZigBee and Bluetooth, can be used for enabling communication between the local processing station and the monitoring terminal, as this communication has a low data rate and spans over a short distance. Thus, the monitoring terminal and the local processing station can be small battery-powered devices, and short-range wireless technologies can preferably be used for enabling this communication.

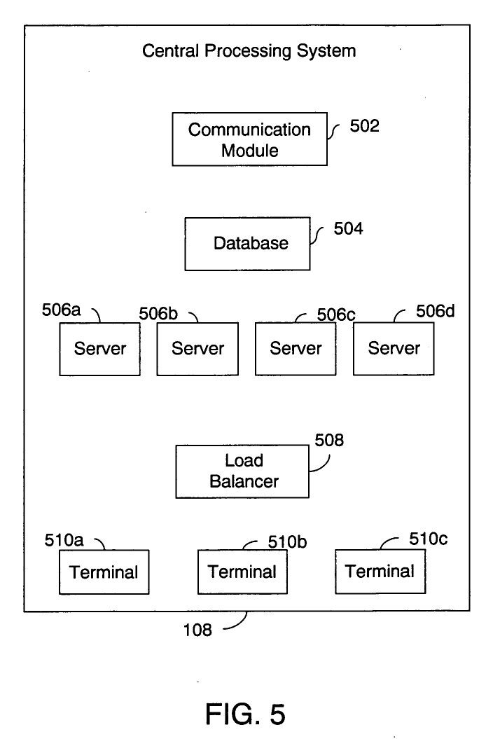

FIG. 5 represents central processing system 108, according to an embodiment of the invention. In an embodiment of the invention, central processing system 108 comprises a communication module 502, a database 504, servers 506a, 506b, 506c, and 506d, a load balancer 508, and terminals 510a, 510b and 510c.

Communication module 502 facilitates communication between central processing system 108 and other components of remote patient monitoring and caring system 100, such as monitoring terminals, local processing systems, and remote access stations. This communication includes reception and transmission of the real-time physiological parameters and their analysis, analyzed historical medical data and messages. The messages include, but are not limited to, text, audio and video messages, and the like.

Database 504 contains the real-time physiological parameters of each patient and their corresponding historical medical data. Database 504 is regularly updated with the real-time physiological parameters of each patient. Medical staff is authorized to access patient data from database 504. Further, via remote access station 110, medical staff, relatives and caretakers associated with a particular patient can access the medical data of the patient from outside remote patient monitoring and caring system 100.

Servers 506a-506d perform further analysis of the real-time physiological parameters obtained from the local processing stations. These real-time physiological parameters and their analysis, of each patient are then processed with respect to his/her historical medical data. The medical staff uses these processed real-time physiological parameters and historical medical data to analyze the medical condition of patients, and provide care and assistance, when required. Further, servers 506a-506d update database 504 with the real-time physiological parameters received from the patients, and the results of the analysis performed on these real-time physiological parameters.

The analysis and processing of the data related to all the patients in remote patient monitoring and caring system 100 is performed by servers 506a-506d. This analysis and processing comprises the load on servers 506a-506d. This load is evenly distributed on all servers 506a-506d by load balancer 508. Load balancer 508 is a device that distributes load evenly on servers 506a-506d, so that no particular server is overloaded with analysis and processing. In addition, load balancer 508 also monitors the functioning of each server and replaces malfunctioning or non-functioning servers with properly functioning servers.

The load on central processing system 108 can be calculated by using algorithms for load balancing, well-known in the art.

Terminals 510a-510c are installed in central processing system 108 to provide access to medical staff to view/review a patient's data. Further, the medical staff can communicate the results of the analysis done on the real-time physiological parameters and the historical medical data, to the patient or any authorized person related to the patient.

The communication between the local processing station and the central processing system can be wired or wireless. Some examples of wireless communication used between the local processing station and the central processing system are WiFi and GSM.

The communication between the local processing station and the central processing system is bi-directional. The local processing station sends the real-time physiological parameters, their analysis and the messages from the patient to the central processing system. The central processing system sends the feedbacks and updates, based on the analysis of the physiological parameters, to each patient, via the local processing station. Further, the central processing system also responds to alarm signals sent by the patients who require immediate response or help.

The communication between the central processing system and the remote access station is via the Internet. Further, the communication between the central processing system and the remote access station is via either a private or a public, wired or wireless, network. An example of public wired networks is Public Switched Telephone Network (PSTN). Some examples of public wireless networks are the Internet and GSM.

The communication between the central processing system and the remote access stations is bi-directional. The remote access station can obtain the real-time physiological parameters and historical medical data of patients, from the central processing system. Further, medical staff and authorized persons accessing these parameters and data can provide a feedback on the same, to the central processing system. This feedback can be sent to the patients, via the local processing station.

In an embodiment of the invention, the functioning of the remote patient monitoring and caring system is described by the following example. A sensor for detecting body temperature is attached to a patient. The body temperature data measured by the sensor is sent to the monitoring terminal of the patient. If the patient thinks that the body temperature is higher/lower than a normal value, the patient can send an alarm signal to the medical staff. Next, the body temperature data is sent to the local processing station. The processing module present in the local processing station analyzes the body temperature data and sends an alarm signal to the patient and the medical staff in case the body temperature is above/below an optimum level. Finally, the body temperature data and its analysis are sent to the central processing system. The central processing system processes the body temperature in combination with the relevant medical history of the patient stored in the database of the central processing system. The medical staff present at the central processing system uses this processed body temperature data to analyze the patient's medical condition and to provide medical care and assistance.

In various embodiments of the invention, the communication between the various components of the remote patient monitoring and caring system is secure, preventing any intentional or unintentional interference to the data and information being exchanged between various components of the remote patient monitoring and caring system. For example, the various wireless data channels are segregated from each other, thus protecting the data of one patient from that of other patients. In addition, proper authentication and encryption of the information and data is done. Further, the system provides security of data transmission between the various components. This security is enabled by using a unique identifier for each sensor and each monitoring terminal. The unique identifier for each sensor is pre-configured to a corresponding monitoring terminal, a corresponding local processing station and a corresponding central processing system, the sensor is connected to. Similarly, the unique identifier for each monitoring terminal is pre-configured to a corresponding local processing station and a corresponding central processing system, the monitoring terminal is connected to. The security settings of the remote patient monitoring and caring system can be dynamically configured by one or more of its components.

In the present invention, each high-level component of the remote patient monitoring and caring system is a manager of its lower-level components. A manager is capable of controlling the functionalities and other operational parameters essential for the working of the component it manages. For example, in an embodiment of the invention, the local processing station controls the functioning of the sensor, via the monitoring terminal. These commands dynamically alter the action of the sensor, according to certain pre-defined rules. In another embodiment of the invention, the central processing system can control the functioning of the sensor, via the local processing station and the monitoring terminal. In yet another embodiment of the invention, the sensor itself can dynamically alter its action, with the help of the sensor control module present in it. However, the functionalities of the local processing station and the central processing system cannot be controlled by the sensor and the monitoring terminal.

Further, a monitoring terminal is a manager of all the sensors it communicates to, and these sensors needs to register to a monitoring terminal. A local processing station manages the monitoring terminals and each monitoring terminal needs to register to a corresponding local processing station. Further, each local processing station needs to register to a central processing system that manages all the local processing stations registered to it.

When a component registers to its manager, an authentication is done. If the authentication fails, the component cannot communicate with its manager, therefore it cannot participate in any of the function in the system.

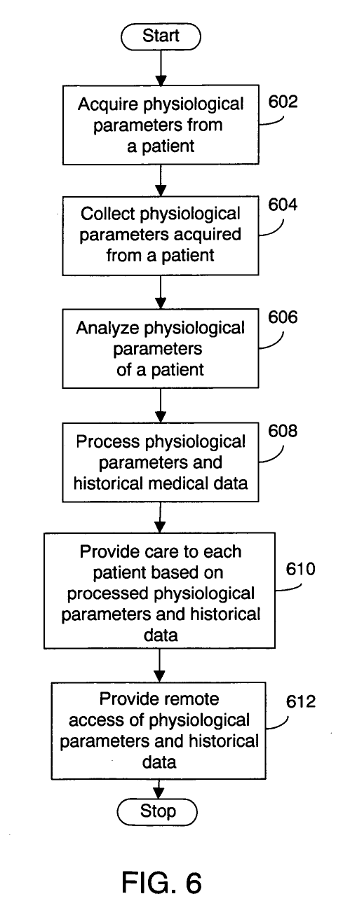

FIG. 6 depicts a flowchart that describes a method for remote patient monitoring and caring, in accordance with an embodiment of the invention.

At step 602, the real-time physiological parameters of a patient are sensed and measured. The sensing is performed by one or more sensors attached to each patient. For example, sensor 102a senses and measures a particular real-time physiological parameter of the patient it is attached to.

At step 604, all the real-time physiological parameters sensed and measured in step 602, from each patient, are collected from each sensor that is attached to the patient. For example, the real-time physiological parameters measured by all sensors 102a-102f attached to a particular patient are collected by monitoring terminal 104 corresponding to the patient. Further, the collection of real-time physiological parameters includes digitization of data and filtration of noise from the real-time physiological parameters.

At step 606, analysis of the real-time physiological parameters that were digitized and filtered in step 604, from each patient, is carried out. This analysis includes basic trends that are generated from the real-time physiological parameters collected from each patient. For example, local processing station 106a analyzes the real-time physiological parameters sent by various monitoring terminals connected to it.

At step 608, the real-time physiological parameters analyzed in step 606, for each patient, are combined with the historical medical data of that patient and processed. For example, central processing system 108 processes the real-time physiological parameters and the historical medical data of each patient and processes them. The analysis includes comparing the real-time physiological parameters and the historical medical data of the patient and deriving trends for each real-time physiological parameter, of each patient. These trends help the medical staff to judge the condition of the patient and care for the patient, and to provide medical assistance, when required.

At step 610, care is provided to each patient based on the corresponding processed real-time physiological parameters and the historical medical data. Further, the medical staff provides medical assistance to patients, when required, based on this analysis. In an embodiment of the invention, if an alarm is generated by any patient, the medical staff responds immediately to the alarm through the terminals present within the central processing system.

At step 612, remote access is provided to medical staff and authorized persons related to each patient outside the medical facility. For example, the remote access stations provide remote access to medical staff and authorized persons related to each patient, from outside the medical facility where the central processing system is present.

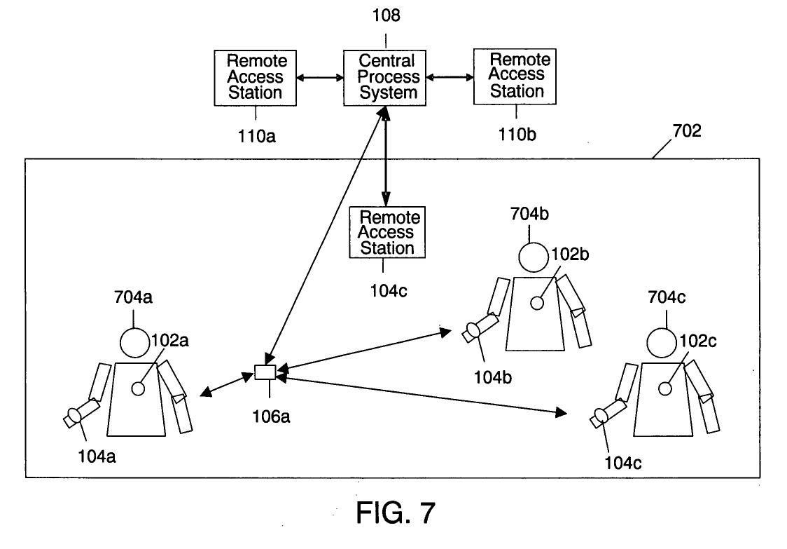

FIG. 7 illustrates the use of remote patient monitoring and caring system 100 for monitoring multiple patients in a community 702, such as a nursing home, in accordance with an embodiment of the invention. In this case, single local processing station 106a is shared by multiple patients. Each patient can only view his/her own data from his/her own monitoring terminal which is connected wirelessly with a sensor on his/her own body. For example, patient 704a can view his/her data from his/her monitoring terminal 104a which is wirelessly connected with sensor 102a on his/her own body. Remote access stations 110a, 110b and 110c can be used by medical staff present within or outside community 702. In an embodiment of the invention, patients 704a, 704b, and 704c use personal local processing stations.

In an embodiment of the invention, a single monitoring terminal can be used to collect real-time physiological parameters from multiple patients. For example, such a monitoring terminal can be wirelessly connected to one or more sensors attached to one or more patients, and placed in a location easily accessible to the several patients. The common monitoring terminal collects data from the various sensors, and transmits the processed data to a local processing station for further handling, wherein the local processing station may be located at or near the common monitoring terminal.

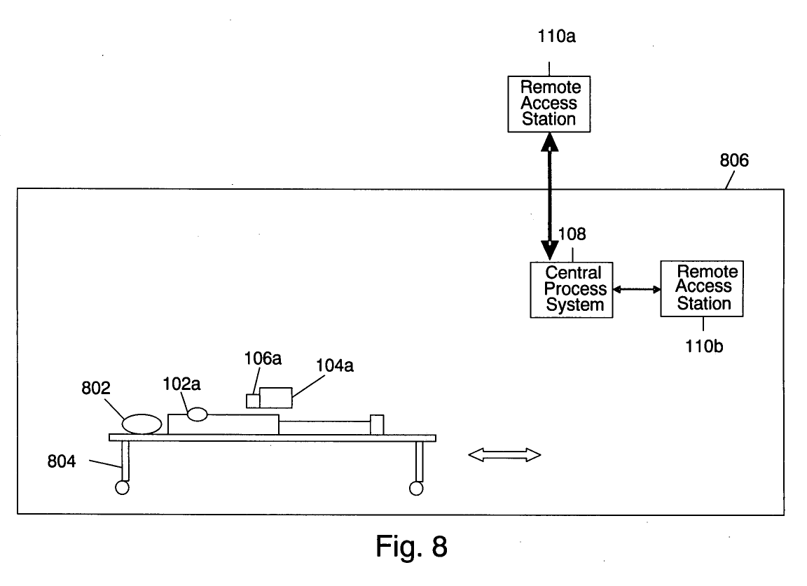

FIG. 8 illustrates the use of remote patient monitoring and caring system 100 for monitoring a patient 802 who is carried on a patient trolley 804, and is in a surgical room 806, in accordance with an embodiment of the invention. Patient 802 is wearing sensor 102a. Monitoring terminal 104a and local processing station 106a are mounted on patient trolley 804 so that a nurse beside the bed can monitor patient 802 through the monitoring terminal 104a. Local processing station 106a communicates with central processing system 108 via wireless communication.

The various embodiments of the invention have the advantage that the distributed architecture of the remote patient monitoring and caring system allows the various functionalities of the system to be intelligibly distributed among its components. This distributed architecture makes the remote patient monitoring and caring system easy to use, maintain, extend and scale. The security systems used in the present invention makes the remote monitoring and caring system safe to use. Further, the system allows bi-directional communication between all the components of the system. The bi-directional communication enables the medical staff to take care of the patients, and provide medical assistance, whenever required. This is not possible in a uni-directional communication, wherein the medical staff can only monitor the patient's condition but cannot provide care to the patient, using the communication system. The system provides for the measurement of all types of real-time physiological parameters, giving way to a well-equipped monitoring system for patients. Finally, the above system can be used not only in hospitals and clinics but also at homes and other non-medical facilities.

The system for, as described in the present invention or any of its components, may be embodied in the form of a computer system. Typical examples of a computer system includes a general-purpose computer, a programmed microprocessor, a micro-controller, a peripheral integrated circuit element, and other devices or arrangements of devices that are capable of implementing the steps that constitute the method of the present invention.

The computer system comprises a computer, an input device, a display unit and the Internet. The computer further comprises a microprocessor. The microprocessor is connected to a communication bus. The computer also includes a memory. The memory may include Random Access Memory (RAM) and Read Only Memory (ROM). The computer system further comprises a storage device. The storage device can be a hard disk drive or a removable storage drive such as a floppy disk drive, optical disk drive, etc. The storage device can also be other similar means for loading computer programs or other instructions into the computer system. The computer system also includes a communication unit. The communication unit allows the computer to connect to other databases and the Internet through an I/O interface. The communication unit allows the transfer as well as reception of data from other databases. The communication unit may include a modem, an Ethernet card, or any similar device, which enables the computer system to connect to databases and networks such as LAN, MAN, WAN and the Internet. The computer system facilitates inputs from a user through input device, accessible to the system through I/O interface.

The computer system executes a set of instructions that are stored in one or more storage elements, in order to process input data. The storage elements may also hold data or other information as desired. The storage element may be in the form of an information source or a physical memory element present in the processing machine.

The set of instructions may include various commands that instruct the processing machine to perform specific tasks such as the steps that constitute the method of the present invention. The set of instructions may be in the form of a software program. Further, the software may be in the form of a collection of separate programs, a program module with a larger program or a portion of a program module, as in the present invention. The software may also include modular programming in the form of object-oriented programming. The processing of input data by the processing machine may be in response to user commands, results of previous processing or a request made by another processing machine.

While the foregoing is directed to embodiments of the present invention, other and further embodiments of the invention may be devised without departing from the basic scope thereof, and the scope thereof is determined by the claims that follow.

1. A system for remote patient monitoring and caring, the system having a distributed architecture, the system comprising:
at least one sensor for acquiring real-time physiological parameters from a patient;
at least one monitoring terminal for collecting the real-time physiological parameters of the patient acquired by one or more sensors;
at least one local processing station for analyzing the real-time physiological parameters collected by one or more monitoring terminals; and
at least one central processing system for processing the analyzed real-time physiological parameters and caring for the patient based on the processed parameters and historical medical data of the patient, wherein the care comprises sending audio, video and text messages.

2. The system according to claim 1 further comprises at least one remote access station for providing access to the real-time physiological parameters and the historical medical data of the patient, wherein the real-time physiological parameters and the historical medical data are stored in the central processing system.

3. The system according to claim 1, wherein the monitoring terminal collects real-time physiological parameters for one or more patients.

4. The system according to claim 1, wherein each monitoring terminal comprises:
a first transceiver for enabling communication between a monitoring terminal and one or more sensors;
a second transceiver for enabling communication between the monitoring terminal and a local processing station;
a control module for formatting the received real-time physiological parameters and generating messages for one or more sensors, the messages being commands required to alter parameters required for the operation of one or more sensors; and
a user-interface for enabling communication between patients and the medical staff.

5. The system according to claim 4, wherein each monitoring terminal further comprises an alarm-generating module for generating an alarm signal when the patient needs immediate help.

6. The system according to claim 1, wherein parameters required for the operation of each sensor can be dynamically altered according to pre-defined rules, by a component selected from the group consisting of the sensor itself, local processing stations, and central processing systems.

7. The system according to claim 1, wherein each local processing station comprises:
a third transceiver for enabling communication between a local processing station and one or more monitoring terminals;
a processing module for processing the formatted real-time physiological parameters received from one or more monitoring terminals;
a fourth transceiver for enabling communication between the local processing station and a central processing system; and
a storage module for storing patient data temporarily when the data is not being transmitted.

8. The system according to claim 7, wherein the local processing station further comprises a media detection module for detecting the availability and quality of the means for transmission.

9. The system according to claim 1, wherein the central processing system comprises:
a communicating module for communicating with one or more local processing stations, monitoring terminals and remote access stations, including communicating with the monitoring terminal from which an alarm signal has been received;
a database for storing historical medical data of patients;
one or more servers for processing the analyzed real-time physiological parameters received from one or more local processing stations, and the stored historical medical data of each patient;
one or more terminals for providing access to patient's data to medical staff, wherein the medical staff views and reviews the patient's data and cares for the patients via the central processing system; and
a load balancer for balancing the load on the servers, the load comprising the analysis of patients' data.

10. The system according to claim 1, wherein the communication between the sensors, the monitoring terminals, the local processing stations, the central processing systems, and remote access stations is bi-directional, and one of the group comprising a wired communication system and a wireless communication system.

11. The system according to claim 1, wherein the communication between the sensors, the monitoring terminals, the local processing stations, the central processing systems, and the remote access stations is secure.

12. The system according to claim 1, wherein the communication between the sensor and the monitoring terminal, is established via one of the group comprising a wired communication system and a wireless communication system, the wireless communication being a short range, low data rate communication technology.

13. The system according to claim 1, wherein the communication between the local processing station and the central processing system, is established via one selected from a group comprising a wired communication system and a long range wireless communication system.

14. The system according to claim 1, wherein the communication between monitoring terminal and the local processing station is established via one of the group comprising a short range wireless communication system and a long range wireless communication system.

15. The system according to claim 1, wherein the communication between the remote access station and the central processing system, is established via one of the group comprising a wired communication system and a wireless communication system.

16. The system according to claim 1, wherein each sensor is registered with a corresponding monitoring terminal, each monitoring terminal is registered with a corresponding local processing station and each local processing station is registered with a corresponding central processing system.

17. The system according to claim 1, wherein the system embodies a method for remote patient monitoring and caring, the method comprising the steps of:
sensing real-time physiological parameters of a patient, wherein the sensing is performed by one or more sensors, each sensor being registered with a corresponding monitoring terminal;
collecting, formatting and digitizing the real-time physiological parameters of the patient, wherein the collecting, formatting and digitizing is performed by the monitoring terminal, each monitoring terminal being registered with a corresponding local processing station;
analyzing the real-time physiological parameters of the patient, wherein the analysis is performed by the local processing station, the local processing station being capable of communicating with one or more monitoring terminals and with the central processing system, each local processing station being registered with a corresponding central processing station;
processing the analyzed real-time physiological parameters and the historical medical data of the patient, wherein the processing is performed by the central processing system;
caring for the patient based on the processed real-time physiological parameters and historical medical data of the patient, wherein the caring is performed by the central processing system, the central processing system being capable of communicating with one or more monitoring terminals, one or more local processing stations, and one or more remote access stations; and
accessing the real-time physiological parameters and the historical medical data of the patient, wherein the accessing is performed by one or more remote access stations.

18. A method for remote patient monitoring and caring, the method comprising the steps of:
sensing real-time physiological parameters of a patient, wherein the sensing is performed by one or more sensors, each sensor being registered with a corresponding monitoring terminal;
collecting, formatting and digitizing the real-time physiological parameters of the patient, wherein the collecting, formatting and digitizing is performed by a monitoring terminal, each monitoring terminal being registered with a corresponding local processing station;
analyzing the real-time physiological parameters of the patient, wherein the analysis is performed by a local processing station, the local processing station being capable of communicating with one or more monitoring terminals and with the central processing system, each local processing station being registered with a corresponding central processing station;
processing the analyzed real-time physiological parameters and historical medical data of the patient, wherein the processing is performed by a plurality of servers present within the central processing system;
caring for the patient based on the processed real-time physiological parameters and historical medical data of the patient, wherein the caring is performed by the central processing system, the central processing system being capable of communicating with one or more monitoring terminals, one or more local processing stations, and one or more remote access stations, the caring comprising sending audio, video and text messages, and providing medical assistance to the patient; and
providing access to the real-time physiological parameters and the historical medical data of the patient, wherein the access is provided through one or more remote access stations.

19. A method for remote patient monitoring and caring, the method comprising the steps of:
sensing real-time physiological parameters of a patient;
collecting, formatting and digitizing the real-time physiological parameters of the patient;
analyzing the real-time physiological parameters of the patient;
processing the analyzed real-time physiological parameters and the historical medical data of the patient; and
caring for the patient based on the processed real-time physiological parameters and the historical medical data of the patient, wherein the care comprises sending audio, video and text messages.

20. The method according to claim 19 further comprises providing remote access to the patients' real-time physiological parameters and historical medical data.
